(19) United States
(12) Patent Application Publication
(10) Pub. No.: US 2023/0355917 A1
(43) Pub. Date: Nov. 9, 2023

(54) Harmonic Capsule

(71) Applicant: Gail Lynn, Platteville, CO (US)

(72) Inventor: Gail Lynn, Platteville, CO (US)

(21) Appl. No.: 18/310,531

(22) Filed: May 1, 2023

Related U.S. Application Data
(60) Provisional application No. 63/337,939, filed on May 3, 2022.

(51) Int. Cl. A61M 21/00

(52) U.S. Cl. CPC A61M 21/0094; A61M 2021/0016; A61M 2021/0044; A61M 2021/0027

(57) ABSTRACT

A harmonic capsule may include a wooden frame that has a base portion, a wall portion both of which can support a top portion, the wall portion includes a left wall, a right wall, and a rear wall. A harmonic capsule may include alignment dowels configured to align the base portion with the wall portion and the wall portion with the top portion, and may include a plurality of fastening elements configured to removable fasten the left wall with the rear wall, the rear wall with the right wall, the base portion with the wall portion, and the top portion with the wall portion. A harmonic capsule may include at least three stabilizing feet configured to removable couple to the base portion.

### CROSS REFERENCE TO RELATED APPLICATIONS

This Application claims priority to U.S. Provisional Patent Application No. 63/337,939 filed on May 3, 2022, entitled “HARMONIC CAPSULE,” which is hereby incorporated herein by reference in its entirety.

### TECHNICAL FIELD

The present disclosure relates to stress and anxiety reduction by controlling a user's exposure to light, sound, and aromatherapy.

### BACKGROUND

Light, sound, and smell are sensory stimuli that can have profound effects on an individual's well-being. It is well understood that these stimuli can be modified, reduced, or exogenously supplemented to manipulate a stress response in a living organism. Controlling exposure to such stimuli can be accomplished through a variety of means, including costly sensory deprivation chambers that require large amounts of synthetic, man-made materials. Additionally, large, complicated modules designed to modulate exposure to various stimuli require complex manufacturing and specialized knowledge to construct.

The present disclosure includes embodiments directed towards controlling exposure of light, sound, and smell to a user in an enclosed environment.

### SUMMARY

According to some embodiments of the present disclosure, a harmonic capsule, (and/or meditation pod, and/or stress reduction chamber) can be used to control exposure to various thermodynamic, electromagnetic, and or mass transfer phenomena. As described herein, the methods and apparatus of the harmonic capsule and its embodiments can be implemented to control exposure to light, sound, and smell for a human, animal, plant, trees, food, jewelry, crystals, or other physical object, when placed into the interior of a harmonic capsule.

In some aspects, the techniques described herein relate to a stress reduction chamber including a wooden frame that has a base portion configured to couple with one or more interconnectable wall portions configured to also couple and support a top portion. The wall portion can include a left wall, a right wall, a rear wall, at least one door, and one or more alignment dowels configured to align the base portion with the wall portion and the wall portion with the top portion. There can also be a plurality of fastening elements configured to removably fasten the left wall with the rear wall, the rear wall with the right wall, the base portion with the wall portion, and the top portion with the wall portion. Furthermore, in some embodiments, the stress reduction chamber includes at least three stabilizing feet configured to removable couple, and level the chamber, to the base portion.

In some aspects, the techniques described herein relate to a stress reduction chamber wherein the door can further include two door elements that each include magnetic closure elements to secure the door element in a closed position. The two door elements each have a rotating portion configured to rotatably communicate with the top portion and the base portion, and an opening portion that has an inside wooden handle, with a wood veneer, and is configured to accommodate the magnetic closure elements where the wood veneer houses the magnetic closure elements. Additionally, the two door elements can maintain a closed position where the opening portion of each of the two door elements are substantially aligned and parallel, and the magnetic closure elements are configured to communicate between the two door elements, in some cases. The two door elements can also maintain an open position where the opening portion of each door elements are configured to be aligned with an opening to accommodate at least the width of a wheelchair.

In some aspects, the techniques described herein relate to a stress reduction chamber where the plurality of fastening elements is configured to be operable by a users' hands without tools. In some cases, each of the plurality of fastening elements further includes a rod assembly and a multipoint latch; where the multipoint latch is operable by the users' hands and communicate with the rod assembly; wherein the rod assembly is configured to communicate simultaneously with at least the wall portion and the base portion.

In some aspects, the techniques described herein relate to a stress reduction chamber wherein the top portion further includes an aromatherapy module with a grille, additionally, the module can have a diffuser that is designed to point downward into the stress reduction chamber. The diffuser can be configured to diffuse an aromatherapy solution through the grille and into an interior portion of the stress reduction chamber, mixing inside the chamber.

In some aspects, the techniques described herein relate to a stress reduction chamber wherein the top portion includes a wireless capable speaker system. The wireless capable speaker system is configured to deliver one or more audio elements to the interior portion of the stress reduction chamber via a speaker directed into the chamber. In some cases, the wireless capable speaker system is configured to project the audio element towards the wall portion of the stress reduction chamber to bounce the audio element off the wall portion of the stress reduction chamber back into the center of the interior portion of the stress reduction chamber. In some cases, the wireless capable speaker is capable of connecting to a network and can be controlled via the user's hand-held electronic device.

As used herein “audio element” and “audio elements” can refer to, but is not limited to: audible soundwaves playable via a speaker, and can include soundwaves not detectable by human physiology, and further in some cases pressure waves at frequencies known to have ergogenic, or any other physiological, effects on the human body, including, but not limited to binaural beats.

In some aspects, the techniques described herein relate to a stress reduction chamber wherein the top portion can include a convection vent configured to accommodate a heat, fluid, and energy transfer from an interior portion of the stress reduction chamber to an exterior of the stress reduction chamber through the convection vent itself.

In some aspects, the techniques described herein relate to a stress reduction chamber wherein the base portion can include one or more lighting arrays configured to display a spectrum of visible and near visible light within the chamber. In some cases, the spectrum of visible and near visible light includes a user selected display occurring over an interval of time, i.e., a light show, that is displayed via the lighting array.

In some aspects, the techniques described herein relate to a method of controlling environmental exposure, which can include: providing a stress reduction chamber designed to internally accommodate an energy recipient; arranging the energy recipient inside the chamber; enclosing the energy recipient inside the stress reduction chamber, the stress reduction chamber including: a wooden frame, a hardwood cladding designed to span between and across the wooden frame, a convection vent designed to allow the transport of air and heat therethrough.

In some cases, the hardwood cladding is configured to encompass the wooden frame interiorly and exteriorly, the hardwood cladding and the wooden frame are also configured to accommodate the convection vent to facilitate communicate air therethrough a top portion of the stress reduction chamber. The hardwood cladding attaches to the interior of the wooden frame to compose the interiorly encompassed wooden frame, which houses the interior portion of the stress reduction chamber. According to some embodiments, the interiorly encompassed wooden frame is configured to enable the passive convective flow of air mass from the interior portion of the stress reduction chamber, through the convection vent thereby exposing the energy recipient to the passive convective flow of the air mass. In some embodiments, the controlling of exposure also includes removing the air mass while convective air currents travel across the energy recipient while the air mass travels from inside the stress reduction chamber through the convective vent, to the outside.

In some aspects, the techniques described herein relate to a method of controlling exposure wherein the convection vent and the interiorly encompassed wooden frame are further configured to accommodate convective energy transfer of a heat generating energy recipient. Including, providing an energy recipient with a heat generating metabolism causes the generation of heat via the heat generating metabolism of the energy recipient. In some cases, the method of controlling exposure further includes transferring the heat from the energy recipient to the convection vent via the interiorly encompassed wooden frame, then removing the heat generated by the energy recipient from the interior portion of the stress reduction chamber through the convection vent.

In some aspects, the techniques described herein relate to a method of controlling exposure which includes providing a wireless capable speaker system configured to integrate into the top portion of the stress reduction chamber. The wireless capable speaker system can include a user interface configured to display a variety of audio elements to a user. The method can also include selecting an audio element via the user interface, and playing the audio element on the wireless capable speaker system. The playing of the audio element is controlled by the user controls audio wave exposure of the energy recipient via the audio element, according to some embodiments. In some cases, the wireless capable speaker system can be integrated into the bottom portion.

In some aspects, the techniques described herein relate to a method of controlling environmental exposure that includes providing a lighting array configured to adapt to a bottom portion of the stress reduction chamber, a wired or wireless receiver configured to communicate with the lighting array and the user interface; selecting a light show on the user interface; and exposing the light show to the energy recipient, thereby controlling exposure to visible light, according to some embodiments. In some cases, the top portion can include a light emitting system.

In some aspects, the techniques described herein relate to a method of controlling environmental exposure that includes providing a rotatably lockable platform configured to couple with the bottom portion of a stress reduction chamber, rotating the rotatably lockable platform from a first position, and locking the rotatably lockable platform to a second position.

In some aspects, the techniques described herein relate to a method of controlling exposure that includes providing an aromatherapy diffuser configured with the top portion of a stress reduction chamber. In some cases, the described method includes a positioning a nozzle configured to physically direct an aroma present in the aromatherapy diffuser, spraying the aroma via the nozzle into the interior portion of the stress reduction chamber, diffusing the aroma around the interior portion of the stress reduction chamber, mixing the aroma in the interior portion of the stress reduction chamber via a convective current in the interior portion of the stress reduction chamber, and exposing the energy recipient to the aroma.

In some aspects, the techniques described herein relate to a meditation pod that includes a plurality of vertical connection members each with at least two connection portions, the at least two connection portions can include a base connection region configured to couple with a base, and a top connection region configured to couple with a top. According to some embodiments, the base of the meditation pod is configured to communicate with the base connection region and accommodate a total load including the plurality of vertical connection members and the top. In some cases, the top connection region of the meditation pod is configured to accommodate a top load made up of the top connection region and the top. In some cases, the plurality of vertical connection members includes a wall section and a door section. The wall section, top and the base each have a perimeter, the wall section has a top connection region perimeter configured to accommodate more than 50% of the top perimeter, thereby making the door section is accommodated by less 50% of the top perimeter, when the door section is closed. The door section is configured to removably communicate with an entry portion of the top perimeter and a step portion of the base perimeter, in an open and closed position. In some cases, the step portion of the base perimeter may include a lighting strip capable of displaying multiple colors.

In some aspects, the techniques described herein relate to a meditation pod, wherein the wall section further includes a plurality of insulation elements configured to couple between and along the plurality of vertical connection members.

In some aspects, the techniques described herein relate to a meditation pod, wherein the plurality of insulation elements further include an inside layer and an outside layer. In some embodiments, the inside layer is configured to absorb acoustic soundwaves in an interior of the meditation pod, and the outside layer is configured to reflect acoustic sound waves. In some cases, the inside layer is configured to reflect acoustic sound waves, and the outside layer is designed to absorb sound waves.

In some aspects, the techniques described herein relate to a meditation pod, further including a wireless speaker configured to couple with the top portion and play one or more sounds directed to the interior of the meditation pod.

In some aspects, the techniques described herein relate to a meditation pod that includes at least one aromatherapy module configured to diffuse a user selected essential oil mixture into the interior of the meditation pod.

In some aspects, the techniques described herein relate to a meditation pod, that includes a power plug with a user-selectable switch configured to select a pre-determined input voltage. By way of example, the pre-determined input voltages may be 120 volts (V), 208V, 220V, and/or 240V.

In some aspects, the techniques described herein relate to a meditation pod, wherein the base further includes a rotatably lockable platform configured to removably couple with a wheelchair ramp configured to accommodate the step portion into the interior of the pod. In some cases, the wheelchair ramp is configured to be removable from the meditation pod when the door of the mediation pod is closed.

Embodiments include one, more, or any combination of the various apparatus and methods described herein. Other features and advantages of the present disclosure will become apparent from the following more detailed description, taken in conjunction with the accompanying, which illustrate, by way of example, the principles of the disclosure.

### BRIEF DESCRIPTION OF THE DRAWINGS

FIG. 1 illustrates a front elevation perspective view of a harmonic capsule, according to embodiments of the present disclosure.

FIG. 2 illustrates a front elevation perspective view of a harmonic capsule, according to embodiments of the present disclosure.

FIG. 3 illustrates an exploded side elevation view of a harmonic capsule, according to embodiments of the present disclosure.

FIG. 4 illustrates a top perspective view of a harmonic capsule, according to embodiments of the present disclosure.

FIG. 5 illustrates a top perspective view of a harmonic capsule, according to embodiments of the present disclosure.

FIG. 6 illustrates an exploded top perspective view of a harmonic capsule, according to embodiments of the present disclosure.

FIG. 7 illustrates an exploded front view of a harmonic capsule, according to embodiments of the present disclosure.

FIG. 8 illustrates a side elevation perspective view of a harmonic capsule, according to embodiments of the present disclosure.

FIG. 9 illustrates an exploded top perspective view of a harmonic capsule, according to embodiments of the present disclosure.

FIG. 10 illustrates flow chart of a method of controlling exposure, according to embodiments of the present disclosure.

FIG. 11 illustrates a front perspective view of a harmonic capsule, according to embodiments of the present disclosure

Corresponding reference characters indicate corresponding parts throughout the several views.

While the disclosure is amendable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

The following detailed description illustrates embodiments of the disclosure and manners by which they can be implemented. Although the best mode of carrying out the present disclosure has been disclosed, those skilled in the art would recognize that other embodiments for carrying out or practicing the present disclosure are also possible.

It should be noted that the terms “first,” “second,” and the like, herein do not denote any order, quantity, or importance, but rather are used to distinguish one element from another. Further, the terms “a” and “an” herein do not denote a limitation of quantity, but rather denote the presence of at least one of the referenced items.

As used herein, the term “wood” can mean, but is not limited to, bamboo, oak, pine, cherry, cedar, walnut, ipe, wood-based polymer, paper composite, or any similar substantially organic structural material.

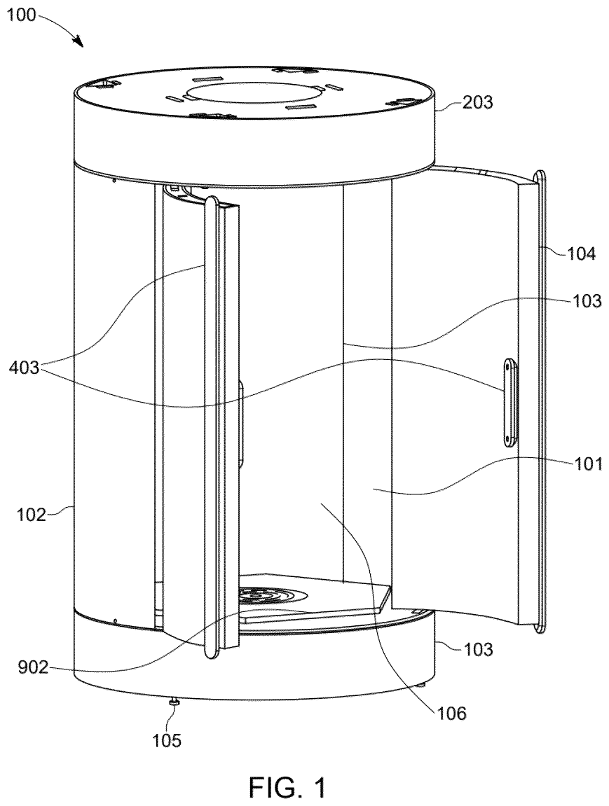

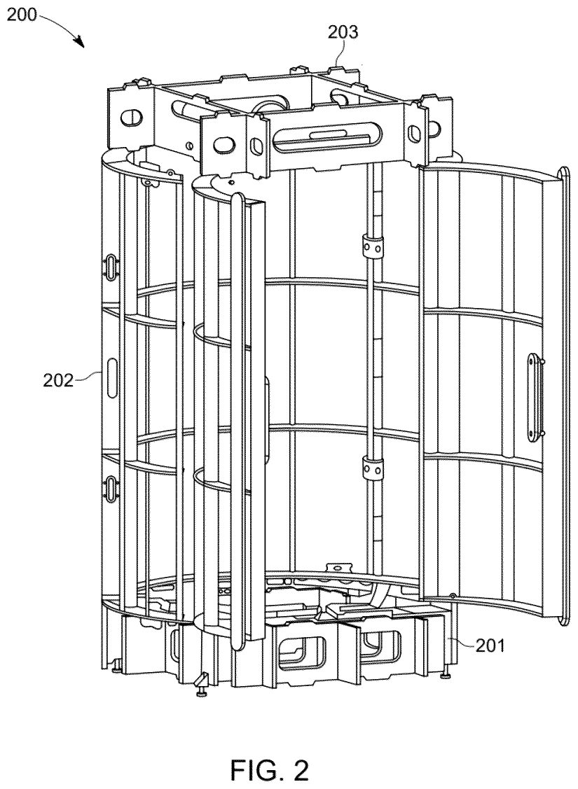

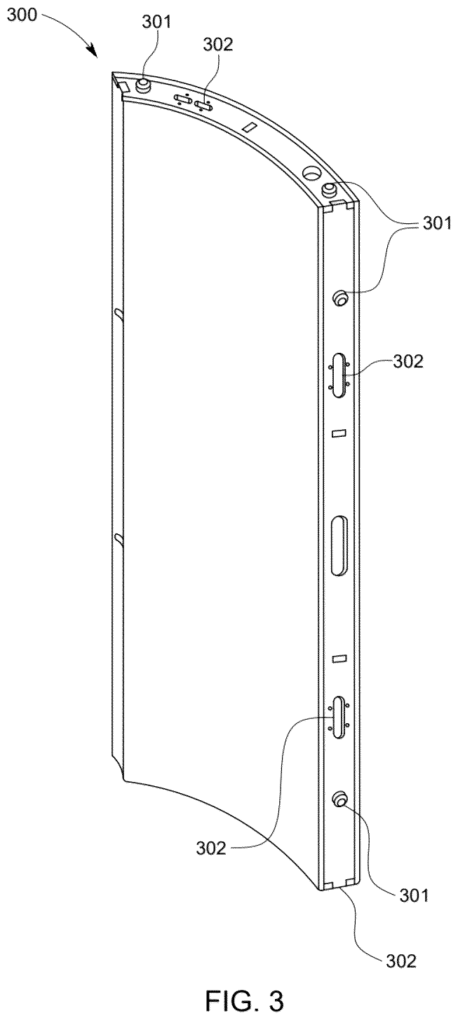

As shown in FIGS. 1, 2, and 3 In some aspects, the techniques described herein relate to a stress reduction chamber 100 including a wooden frame 200 that has a base portion 201 configured to couple with a wall portion 202 configured to also couple and support a top portion 203. The wall portion 202 can include one or more interconnectable walls 300, such as: a left wall 101, a right wall 102, a rear wall 103, at least one door 104, and one or more alignment dowels 301 configured to align the base portion 201 with the wall portion 202 as well as the wall portion 202 with the top portion 203. There can also be a plurality of fastening elements 302 configured to removably fasten the left wall 101 with the rear wall 103, the rear wall 103 with the right wall 102, the base portion 201 with the wall portion 202, and the top portion 203 with the wall portion 202. In some cases, plurality of fastening elements 302 can be, but are not limited to: compression latches, cam latches, push-to-close latches, rotary latches, draw latches, swing handle latches, multipoint latches, entry door latches, and/or electronic latches. Furthermore, in some embodiments, the stress reduction chamber 100 includes at least three stabilizing feet 105 configured to removably couple, and level the stress reduction chamber 100, to the base portion 201.

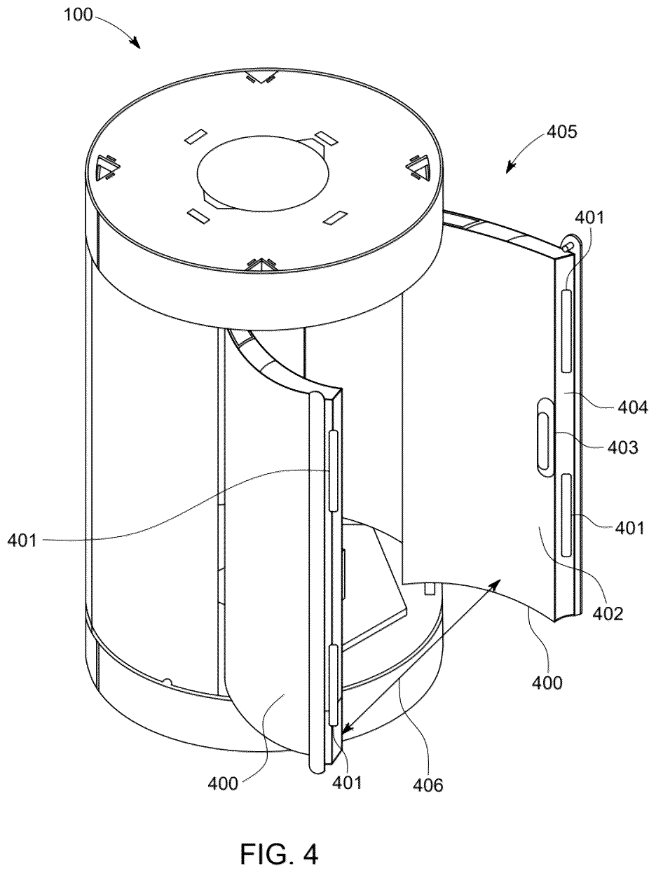

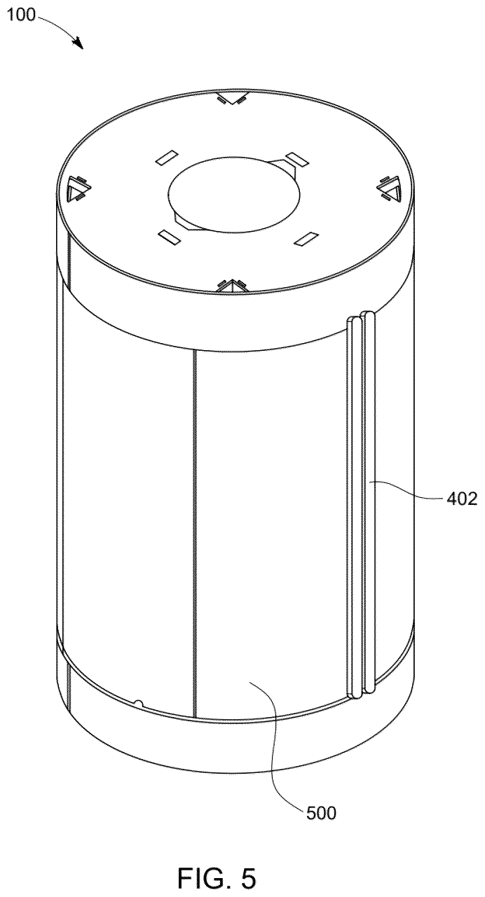

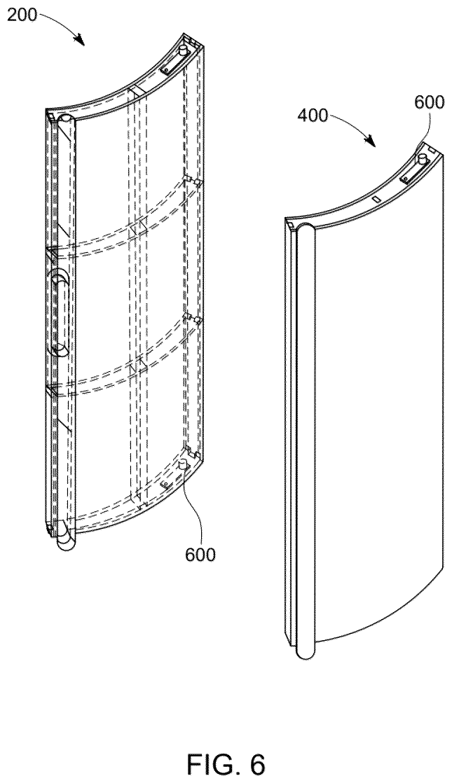

As shown in FIGS. 1, 4, 5, 6, and 7 In some aspects, the techniques described herein relate to a stress reduction chamber 100 wherein the at least one door 104 can further include two door elements 400 that each include closure elements 401 to secure two door elements 400 in a closed position 500. The two door elements 400 each have a rotating portion 600 configured to rotatably communicate 602 with the top portion 203 and the base portion 201, and an opening portion 402 that has an inside wooden handle 403, with a wood veneer 404, and is configured to accommodate the closure elements 401 where the wood veneer 404 houses the closure elements 401. As used herein, the wood veneer 404 can be, but is not limited to a veneer, cladding, skin, or other housing that substantially covers or encompasses an interior frame accomplished through the use of wood-based products. Additionally, the two door elements 400 can maintain a closed position 500 where the opening portion 402 of each of the two door elements are substantially aligned and parallel, and the closure elements 401 are configured to communicate between the two door elements 400, in some cases. The two door elements 400 can also maintain an open position 405 where the opening portion 402 of each door elements are configured to be aligned with an opening 406 to accommodate at least the width of a standard ADA wheelchair, or 32 inches.

As used herein, “closure elements” can mean, but is not limited to, fasteners, magnets, electromagnetic lock, radio-frequency identification (RFID) closure device, latches, clips, buttons, buckles, or similar fastening closure apparatus.

As Shown in FIGS. 1, 2, and 3 the techniques described herein relate to a stress reduction chamber 100 where the plurality of fastening elements 302 is configured to be operable by a users' hands without the use of tools. In some cases, each of the plurality of fastening elements 302 further includes a rod assembly and a multipoint latch; where the multipoint latch is operable by the users' hands and communicate with the rod assembly; wherein the rod assembly is configured to communicate simultaneously with at least the wall portion 202 and the base portion 201.

As shown in FIGS. 1, 7, and 8 the techniques described herein relate to a stress reduction chamber 100 wherein the top portion 203 further includes a sound and smell module 801 with a grille 701, additionally, the sound and smell module 801 can have a diffuser 802 that is designed to communicate with the grille 701. According to some embodiments, the diffuser 802 can be configured to diffuse an aromatherapy solution through the grille 701 and into an interior portion 106 of the stress reduction chamber 100, where the aroma therapy solution can mix inside the interior portion 106 of the stress reduction chamber 100.

As shown in FIGS. 1, 7, and 8 the techniques described herein relate to a stress reduction chamber wherein the top portion 203 includes a wireless capable speaker system 702, in some cases. As used herein, “wireless” can mean, but is not limited to wireless network protocols, Bluetooth, or comparable ISM radio bands to deliver information wirelessly from a transmitter to a receiver. The wireless capable speaker system 702 is configured to deliver one or more audio element to the interior portion 106 of the stress reduction chamber 100 via a speaker directed into the interior portion 106. In some cases, the wireless capable speaker system 702 is configured to project the audio element towards the wall portion of the stress reduction chamber to bounce the audio element off the wall portion 202 of the stress reduction chamber 100 back into the center of the interior portion 106 of the stress reduction chamber 100.

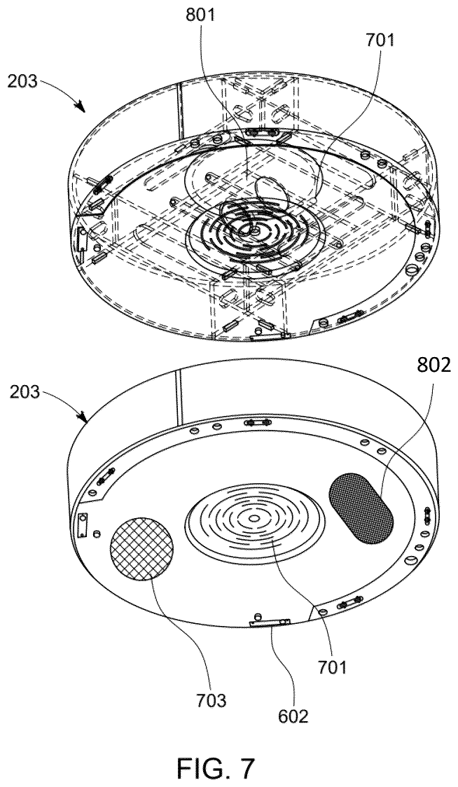

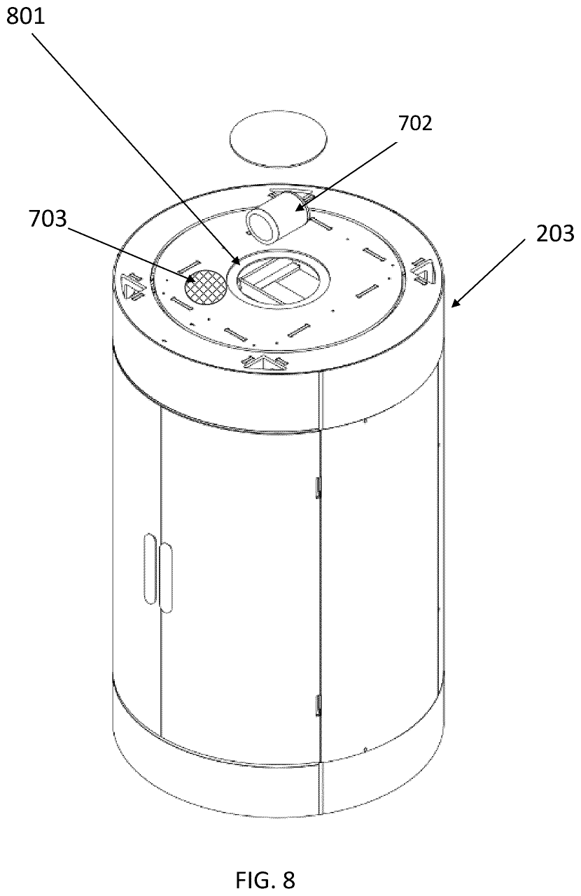

As Shown in FIGS. 7 and 8, the techniques described herein can relate to a stress reduction chamber 100 wherein the top portion 203 can include a convection vent 703 configured to accommodate a heat, fluid, and energy transfer from an interior portion of the stress reduction chamber to an exterior of the stress reduction chamber through the convection vent 703.

According to some embodiments, the convection vent 703 may house a reversible van. The reversible fan can be controlled via a user to cause forced convection of the airmass inside the stress reduction chamber 100, where the air mass of the interior portion 106 is manipulated by the action of the reversible fan. The reversible fan can cause the cooling or heating of the interior portion 106 depending on the temperature differential between the interior portion 106 and the ambient air outside the stress reduction chamber 100.

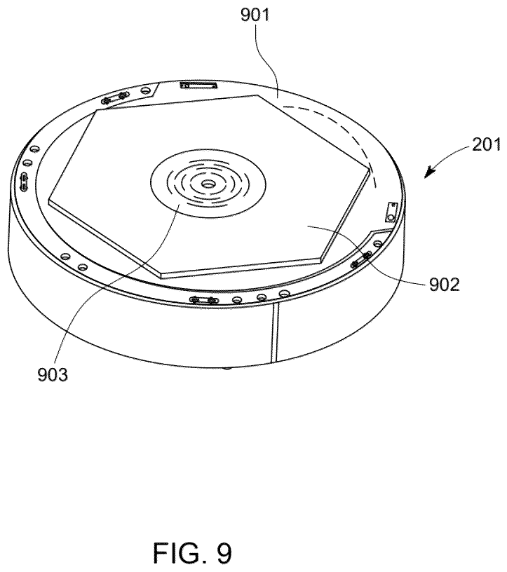

As shown in FIG. 9 according to some embodiments, the techniques described herein relate to a stress reduction chamber 100 wherein the base portion 201 can a lighting array 901 configured to display a spectrum of visible and near visible light within the stress reduction chamber 100. In some cases, the spectrum of visible and near visible light includes a user selected display occurring over an interval of time, i.e., a light show, that is displayed via the lighting array 901. According to some embodiments the lighting array 901 may also extend to the wall portion 202 and/or the top portion 203. In some cases, the stress reduction chamber 100 can include the lighting array 901 configured to adapt to the top portion 203.

As used herein, “lighting array” can mean, but is not limited to, a light emitting diode (LED) strip, fiber optics, infrared heating lights, or other visible or near visible electromagnetic radiation device. In some cases, the lighting array can comprise of a combination of LEDs, infrared heating lights, and/or fiber optics.

As shown in FIG. 9 according to some embodiments, the base portion 201 includes a platform 902. The platform 902 can be configured to rotate and lock via a cam, lever, spring, and wheel system. In some cases, the platform 902 can be lifted, releasing the cam from the lever, and adjusted a new position by rotating the platform 902 and attached wheel to a new position. Then the platform 902 can be lowered causing the spring to engage and the lever catching on the cam. In some cases, the platform is shaped as a polygon, such as a hexagon.

In some cases, the platform 902 also includes a base convection vent 903. The base convection vent 903 can be configured to facilitate the convective heat transfer and mass transfer between the interior portion 106 and the exterior of the stress reduction chamber 100. The base convection vent 903 can also include a reversible fan module configured to be operable by user and in some cases communicate with the convection vent 703 fan. The base convection vent 903 also houses a secondary aromatherapy module, in some cases.

According to some embodiments, the stress reduction chamber can be rotated into a horizontal application, so the user may lie in a prone or supine position while inside the chamber. In such cases, the stress reduction chamber interior platform can be alongside one of the walls, and the doors open in a substantially vertical direction.

According to some embodiments, the stress reduction chamber includes a warning signal, the warning signal can be operated by a user inside the stress reduction chamber. In some cases, the user can operate the warning signal via a hand-held electronic device, such as a smartphone, tablet, personal computer, or user interface (i.e. display) provided in the interior of the stress reduction chamber. The warning signal can be designed to send a notification to a third-party via a network connection, or in some cases an auditory sound emitted from an external warning signal amplifier. In some cases, the operation of the warning signal simultaneously stops the playing of any audio elements and/or light show and notifies the third party via an auditory signal and network notification.

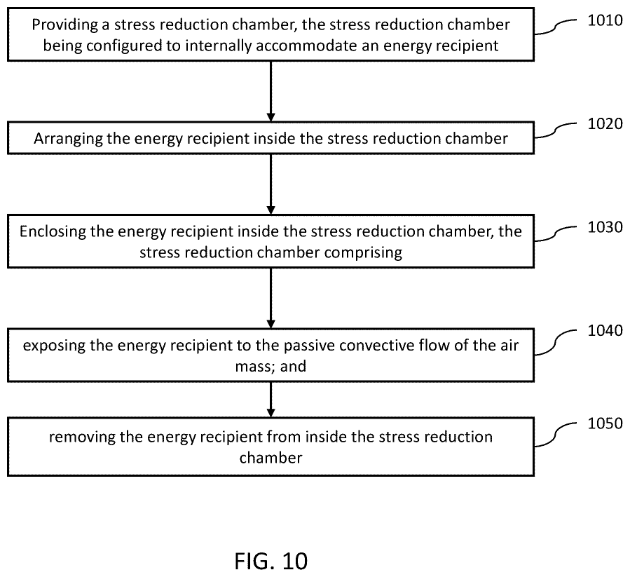

As shown in FIG. 10 in some aspects, the techniques described herein relate to a method of controlling environmental exposure, which can include: providing a stress reduction chamber designed to internally accommodate an energy recipient 1010; arranging the energy recipient inside the chamber 1020; enclosing the energy recipient inside the stress reduction chamber 1030, the stress reduction chamber including: a wooden frame, a hardwood cladding designed to span between and across the wooden frame, a convection vent designed to allow the transport of air and heat therethrough.

In some cases, the hardwood cladding is configured to encompass the wooden frame interiorly and exteriorly, the hardwood cladding and the wooden frame are also configured to accommodate the convection vent to facilitate communicate air therethrough a top portion of the stress reduction chamber. The hardwood cladding attaches to the interior of the wooden frame to compose the interiorly encompassed wooden frame, which houses the interior portion of the stress reduction chamber. According to some embodiments, the interiorly encompassed wooden frame is configured to enable the passive convective flow of air mass from the interior portion of the stress reduction chamber, through the convection vent thereby exposing the energy recipient to the passive convective flow of the air mass 1040. In some embodiments, the controlling of exposure also includes removing the air mass while convective air currents travel across the energy recipient while the air mass travels from inside the stress reduction chamber 1050 through the convective vent, to the outside.

In some aspects, the techniques described herein relate to a method of controlling exposure wherein the convection vent and the interiorly encompassed wooden frame are further configured to accommodate convective energy transfer of a heat generating energy recipient. Whereby, providing an energy recipient with a heat generating metabolism causes the generating of heat via the heat generating metabolism of the energy recipient. In some cases, the method of controlling exposure further includes transferring the heat from the energy recipient to the convection vent via the interiorly encompassed wooden frame, then removing the heat generated by the energy recipient from the interior portion of the stress reduction chamber through the convection vent.

In some aspects, the techniques described herein relate to a method of controlling exposure which includes providing a wireless capable speaker system configured to integrate into the top portion of the stress reduction chamber. The wireless capable speaker system can include a user interface configured to display a variety of audio elements to a user. The method can also include selecting an audio element via the user interface, and playing the audio element on the wireless capable speaker system. The playing of the audio element is controlled by the user controls audio wave exposure of the energy recipient via the audio element, according to some embodiments.

In some aspects, the techniques described herein relate to a method of controlling environmental exposure that includes providing a lighting array configured to adapt to a bottom portion of the stress reduction chamber, a wired or wireless receiver configured to communicate with the lighting array and the user interface; selecting a light show on the user interface; and exposing the light show to the energy recipient, thereby controlling exposure to visible light, according to some embodiments. In some cases, the displaying of audio element is limited by the contemporaneous selection of the light show, where the selection of a particular light show limits the selecting of which audio elements may be selected by the user.

In some aspects, the techniques described herein related to a method of controlling environmental exposure that includes a timing interval, wherein the timing interval is associated with the light show and/or the audio elements. In some cases, the timing interval can be determined by the user input on the wired or wireless receiver. According to some embodiments, the wired or wired receiver can communicate with a hand-held electronic device such as a smart phone, tablet, or personal computer. In some aspects, the wired or wireless receiver is capable of connecting to a network and communicating with the hand-held electronic device.

In some aspects, the techniques described herein relate to a method of controlling environmental exposure that includes selecting a timing interval on the hand-held electronic device, and the timing interval determining the length of time the light show and/or the audio elements will occur in the stress reduction chamber.

In some aspects, the techniques described herein relate to a method of controlling environmental exposure that includes providing a rotatably lockable platform configured to couple with the bottom portion of a stress reduction chamber, rotating the rotatably lockable platform from a first position, and locking the rotatably lockable platform to a second position.

In some aspects, the techniques described herein relate to a method of controlling exposure that includes providing an aromatherapy diffuser configured with the top portion of a stress reduction chamber. In some cases, the described method includes a positioning a nozzle configured to physically direct an aroma present in the aromatherapy diffuser, spraying the aroma via the nozzle into the interior portion of the stress reduction chamber, diffusing the aroma around the interior portion of the stress reduction chamber, mixing the aroma in the interior portion of the stress reduction chamber via a convective current in the interior portion of the stress reduction chamber, and exposing the energy recipient to the aroma.

In some cases, the method of controlling exposure may further include the selection of an aroma based on a pre-selected variety. According to some embodiments the selection of a specific aroma from the pre-selected variety will then limit the playing of the audio element to a limited variety of available audio elements, and further limit the selection of the light show. In some cases, the user may select from a pre-determined list of exposure goals: selecting an exposure goal will cause the automatic, simultaneous playing of the audio elements and diffusing of the aroma.

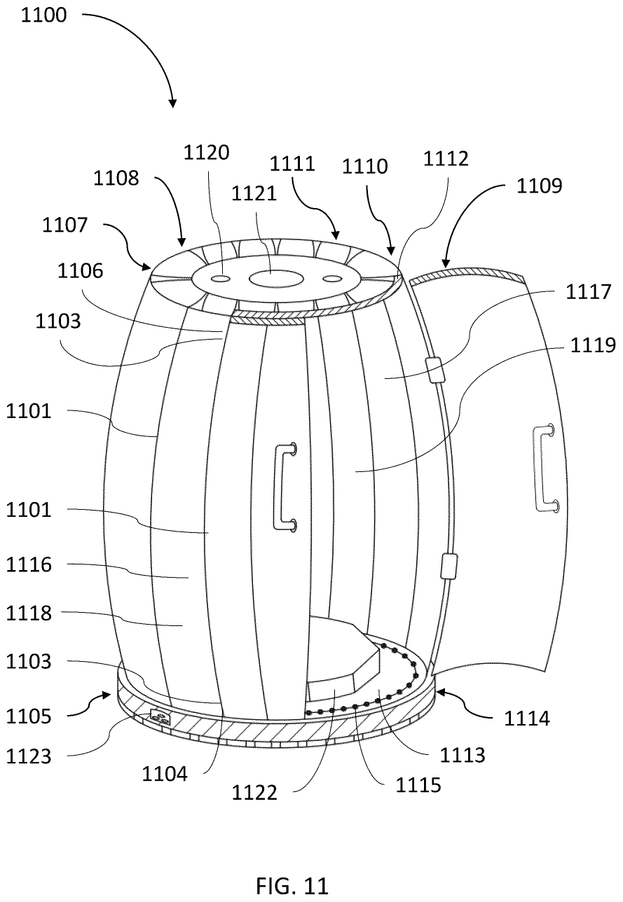

As shown in FIG. 11, in some aspects, the techniques described herein relate to a meditation pod 1100 that includes a plurality of vertical connection members 1101 each with at least two connection portions 1103, the at least two connection portions 1103 can include a base connection region 1104 configured to couple with a base 1105, and a top connection region 1106 configured to couple with a top 1107. According to some embodiments, the base 1105 of the meditation pod 1100 is configured to communicate with the base connection region 1104 and accommodate a total load including the plurality of vertical connection members 1101 and the top 1107. In some cases, the top connection region 1106 of the meditation pod 1100 is configured to accommodate a top load made up of the top connection region 1106 and the top 1107.

As shown in FIG. 11, in some cases, the plurality of vertical connection members 1101 includes a wall section 1108 and a door section 1109. The wall section 1108, top 1107 and the base 1105 each have a perimeter, the wall section 1108 has a top connection region perimeter 1110 configured to accommodate more than 50% of the top perimeter 1111, thereby making the door section 1109 accommodated by less 50% of the top perimeter 1111, when the door section 1109 is closed. The door section 1109 is configured to removably communicate with an entry portion 1112 of the top perimeter 1111 and a step portion 1113 of the base perimeter 1114, in an open and closed position. In some cases, the step portion 1113 of the base perimeter 1114 may include a lighting strip 1115 capable of displaying multiple colors. In some cases, the meditation pod 1100 can include a lighting strip 1115 on the top 1107.

As used herein, “lighting strip” can mean, but is not limited to, a light emitting diode (LED) strip, fiber optics, infrared heating lights, or other visible or near visible electromagnetic radiation device. In some cases, the lighting array can comprise of a combination of LEDs, infrared heating lights, and/or fiber optics.

As shown in FIG. 11, in some aspects, the techniques described herein relate to a meditation pod 1100, wherein the wall section 1108 further includes a plurality of insulation elements 1116 configured to couple between and along the plurality of vertical connection members 1101.

As shown in FIG. 11, in some aspects, the techniques described herein relate to a meditation pod 1100, wherein the plurality of insulation elements 1116 further include an inside layer 1117 and an outside layer 1118. In some embodiments, the inside layer 1117 is configured to absorb acoustic soundwaves in an interior 1119 of the meditation pod 1100, and the outside layer 1118 is configured to reflect acoustic sound waves. In some cases, the inside layer 1117 is configured to reflect acoustic sound waves, and the outside layer 1118 is designed to absorb sound waves.

As shown in FIG. 11, in some aspects, the techniques described herein relate to a meditation pod 1100, further including a wireless capable speaker system 1120 configured to couple with the top 1107 and play one or more sounds directed to the interior 1119 of the meditation pod 1100.

As shown in FIG. 11, in some aspects, the techniques described herein relate to a meditation pod 1100 that includes at least one aromatherapy module 1121 configured to diffuse a user selected essential oil mixture into the interior 1119 of the meditation pod 1100.

As shown in FIG. 11, in some aspects, the techniques described herein relate to a meditation pod 1100, that includes a power plug 1123 with a user-selectable switch configured to select a pre-determined input voltage. By way of example, the pre-determined input voltages may be 120 volts (V), 208V, 220V, and/or 240V.

As shown in FIG. 11, in some aspects, the techniques described herein relate to a meditation pod 1100, wherein the base further includes a rotatably lockable platform 1122 configured to removably couple with a wheelchair ramp configured to accommodate the step portion 1113 into the interior 1119 of the meditation pod 1100. In some cases, the wheelchair ramp is configured to be removable from the meditation pod 1100 when the door of the meditation pod 1100 is closed. In some cases, the rotatably lockable platform 1122 is hexagonally shaped.

Embodiments include one, more, or any combination of the various apparatus and methods described herein. Other features and advantages of the present disclosure will become apparent from the following more detailed description, taken in conjunction with the accompanying, which illustrate, by way of example, the principles of the disclosure.

The examples mentioned above are merely used for illustrative purposes and not meant to be limitations of the present disclosure.

What is claimed is:

1. A stress reduction chamber comprising:
a wooden frame comprising:
a base portion configured to couple with a wall portion configured to couple and support a top portion;
Wherein the wall portion comprises:
a left wall,
a right wall,
a rear wall,
at least one door,
alignment dowels configured to align the base portion with the wall portion and the wall portion with the top portion;
a plurality of fastening elements configured to removable fasten the left wall with the rear wall, the rear wall with the right wall, the base portion with the wall portion, and the top portion with the wall portion; and
at least three stabilizing feet configured to removable couple to the base portion.

2. The stress reduction chamber of claim 1 wherein the at least one door further comprises two door elements each comprising closure elements;
wherein the two door elements each have a rotating portion configured to rotatably communicate with the top portion and the base portion, and an opening portion comprising an inside wooden handle, a wood veneer, and is configured to accommodate the closure elements where the wood veneer houses the closure elements;
wherein the two door elements have a closed position where the opening portion of each of the two door elements are substantially aligned and the closure elements are configured to communicate between the two door elements, and an open position where the opening portion of each door elements are configured to be aligned with an opening to accommodate at least a wheelchair.

3. The stress reduction chamber of claim 1 wherein the plurality of fastening elements is configured to be operable by a users' hands, wherein each of the plurality of fastening elements further comprises a rod assembly and a multi point latch;
wherein the multipoint latch is configured to be operable by the users' hands and communicate with the rod assembly;
wherein the rod assembly is configured to communicate simultaneously with at least the wall portion and the base portion.

4. The stress reduction chamber of claim 1 wherein the top portion further comprises:
An aromatherapy module comprising a grille, and at least one diffuser configured to diffuse into the stress reduction chamber;
wherein the at least one diffuser in configured to diffuse an aromatherapy solution through the grille and into an interior portion of the stress reduction chamber.

5. The stress reduction chamber of claim 4 wherein the top portion further comprises:
a wireless capable speaker system configured to deliver an audio element to the interior portion of the stress reduction chamber;
wherein the wireless capable speaker system is configured to project the audio element towards the wall portion of the stress reduction chamber to bounce the audio element off the wall portion of the stress reduction chamber into the interior portion of the stress reduction chamber.

6. The stress reduction chamber of claim 1 wherein the top portion further comprises: a convection vent configured to accommodate a heat, fluid, and energy transfer from an interior portion of the stress reduction chamber to an exterior of the stress reduction chamber.

7. The stress reduction chamber of claim 1 wherein the base portion further comprises: a light emitting array configured to display a spectrum of visible and near visible light;
wherein the spectrum of visible and near visible light comprises a user selected light show.

8. A method of controlling exposure, comprising:
providing a stress reduction chamber, the stress reduction chamber being configured to internally accommodate an energy recipient;
arranging the energy recipient inside the stress reduction chamber;
enclosing the energy recipient inside the stress reduction chamber, the stress reduction chamber comprising:
a wooden frame,
a hardwood cladding,
a convection vent;
wherein the hardwood cladding is configured to encompass the wooden frame interiorly and exteriorly, the hardwood cladding and the wooden frame also configured to accommodate the convection vent that is configured to communicate air therethrough a top portion of the stress reduction chamber; and
an interiorly encompassed wooden frame that substantially comprises an interior portion of the stress reduction chamber;
wherein the interiorly encompassed wooden frame is configured to enable a passive convective flow of an air mass in the interior portion of the stress reduction chamber;
exposing the energy recipient to the passive convective flow of the air mass; and
removing the energy recipient from inside the stress reduction chamber.

9. The method of controlling exposure of claim 8 wherein the convection vent and the interiorly encompassed wooden frame are further configured to accommodate convective energy transfer of a heat generating energy recipient;
providing and energy recipient with a heat generating metabolism;
generating a heat via the heat generating metabolism;
transferring the heat from the energy recipient to the convection vent via the interiorly encompassed wooden frame; and
removing the heat from the interior portion of the stress reduction chamber through the convection vent.

10. The method of controlling exposure of claim 8 further comprising:
providing a wireless capable speaker system configured to integrate into the top portion of the stress reduction chamber, a user interface configured to display a variety of audio elements to a user;
selecting an audio element via the user interface;
playing the audio element on the Wireless capable speaker system; and
exposing the energy recipient to the audio element.

11. The method of controlling exposure of claim 10 further comprising:
providing a light emitting array configured to adapt to a bottom portion of the stress reduction chamber, a wireless receiver configured to communicate with the light emitting array and the user interface;
selecting a light show on the user interface; and
exposing the light show to the energy recipient.

12. The method of controlling exposure of claim 8 further comprising:
providing a rotatably lockable platform configured to couple with a bottom portion;
rotating the rotatably lockable platform; and
locking the rotatably lockable platform.

13. The method of controlling exposure of claim 8 further comprising:
providing an aromatherapy diffuser configured couple with the top portion and comprising a nozzle configured to direct an aroma into the interior portion of the stress reduction chamber;
diffusing the aroma into the interior portion of the stress reduction chamber; and
mixing the aroma in the interior portion of the stress reduction chamber via a convective current in the interior portion of the stress reduction chamber;
exposing the energy recipient to the aroma.

14. A meditation pod comprising:
a plurality of vertical connection members each with at least two connection portions, the at least two connection portions comprising: a base connection region configured to couple with a base, and a top connection region configured to couple with a top;
wherein the base is configured to communicate with the base connection region and accommodate a total load comprising the plurality of vertical connection members and the top;
wherein the top connection region is configured to accommodate a top load comprising the top connection region and the top;
wherein the plurality of vertical connection members comprises a wall section and a door section, the wall section;
wherein the top and the base each have a perimeter, the wall section has a top connection region perimeter configured to accommodate more than 50% of the top perimeter; and
wherein the door section is configured to removably communicate with an entry portion of the top perimeter and a step portion of the base perimeter;
wherein the step portion of the base perimeter comprises a lighting strip.

15. The meditation pod of claim 14, wherein the wall section further comprises a plurality of insulation elements configured to couple between and along the plurality of vertical connection members.

16. The meditation pod of claim 15, wherein the plurality of insulation elements further comprising an inside layer and an outside layer;
wherein the inside layer is configured to absorb acoustic soundwaves in an interior of the meditation pod, and the outside layer is configured to reflect acoustic sound waves.

17. The meditation pod of claim 16, further comprising:
a Wireless speaker configured to couple with the top and play an audio element directed to the interior of the meditation pod.

18. The meditation pod of claim 17, further comprising:
at least one aromatherapy module configured to diffuse a user selected essential oil mixture into the interior of the meditation pod.

19. The meditation pod of claim 18, further comprising:
a power plug with a user-selectable switch configured to select a pre-determined input voltage.

20. The meditation pod of claim 19, wherein the base further comprises:
a rotatably lockable platform configured to removably couple with a wheelchair ramp configured to accommodate the step portion.
